(19) United States
(12) Patent Application Publication
(10) Pub. No.: US 2009/0234324 A1
(43) Pub. Date: Sep. 17, 2009

(54) Co-Access Port Introducer

(76) Inventors: Robert F. Rioux, Ashland, MA (US); Mark Girard, Waltham, MA (US)

Correspondence Address: FAY KAPLUN & MARCIN, LLP, 150 BROADWAY, SUITE 702, NEW YORK, NY, 10038, US

(21) Appl. No.: 12/208,579

(22) Filed: Sep. 11, 2008

Related U.S. Application Data
(60) Provisional application No. 60/973,950, filed on Sep. 20, 2007.

(51) Int. Cl. A61M 5/162

(52) U.S. Cl. 604/513; 604/175

(57) ABSTRACT

A power injection system comprises an introducer needle including a lumen extending therethrough to a non-coring distal tip and an anchoring device extendable laterally from the introducer needle distal tip, the anchoring device including a port contacting surface engaging one of an inner surface of the port and an inner surface of a septum to anchor the introducer needle within a subcutaneous port.

### PRIORITY CLAIM

The present claims priority to U.S. Provisional Patent Application Ser. No. 60/973,950 filed on Sep. 20, 2007 entitled “Co-Access Port Introducer”. The entire disclosure of this application is expressly incorporated herein by reference.

### BACKGROUND

The treatment of chronic diseases often requires repeated and prolonged access to the vascular system. The more frequently these procedures must be repeated, the more impractical and dangerous it becomes to insert and remove a needle from a vein at every session. Thus, patients are often fitted with a semi-permanent catheter which remains in place in communication with the vascular system between sessions. Proximal ends of these catheters which remain accessible (e.g., via subcutaneously implanted ports) are often sealed with valves such as Pressure Actuated Safety Valves (PASV's). These PASV's open only when a fluid pressure within the catheter exceeds a preselected threshold pressure and re-seal when the fluid pressure drops below this threshold pressure.

Most procedures for infusing fluids through such semi-permanent catheters (e.g., delivering chemotherapy agents, antibiotics, drugs and/or blood products) involve low flow rates and injection pressures. However, for certain applications it is necessary to introduce fluid into the catheter at pressure and flow rates higher than those commonly used for such therapeutic infusions. For example, power injection of contrast media used in visualization techniques require flow rates and pressures in excess of those for which most conventional catheter systems and ports are suited. These procedures, commonly referred to as power injection procedures, have typically required a separate, more robust catheter than those used in conventional infusion techniques. If a power injection is carried out via a typical port, the high back pressure generated will often force the needle back out of the port.

### SUMMARY OF THE INVENTION

The present invention is directed to a power injection system comprising an introducer needle including a lumen extending therethrough to a non-coring distal tip and an anchoring device extendable laterally from the introducer needle distal tip, the anchoring device including a port contacting surface engaging one of an inner surface of the port and an inner surface of a septum to anchor the introducer needle within a subcutaneous port.

### BRIEF DESCRIPTION OF DRAWINGS

FIG. 1 is a schematic illustration showing a port, an introducer needle and an anchoring needle according to a first embodiment of the invention; and

FIG. 2 is a schematic illustration showing a port, an introducer needle and an anchoring needle according to a second embodiment of the invention.

### DETAILED DESCRIPTION

The present invention may be further understood with reference to the following description and the appended drawings, wherein like elements are referred to with the same reference numerals. The invention relates to devices used to supply pressurized fluid to a catheter. More specifically, the invention relates to a method and device to connect a power injection device to a catheter through a port, while retaining the device in place and protecting a valve of the catheter from damage.

According to the invention, the port is fitted with a soft septum that can be easily pierced by the needle of a conventional or a power injection syringe while, for power injection procedures, a non-coring needle is used to pierce the septum and an anchoring needle is introduced through the non-coring needle to retain the device in place during the procedure.

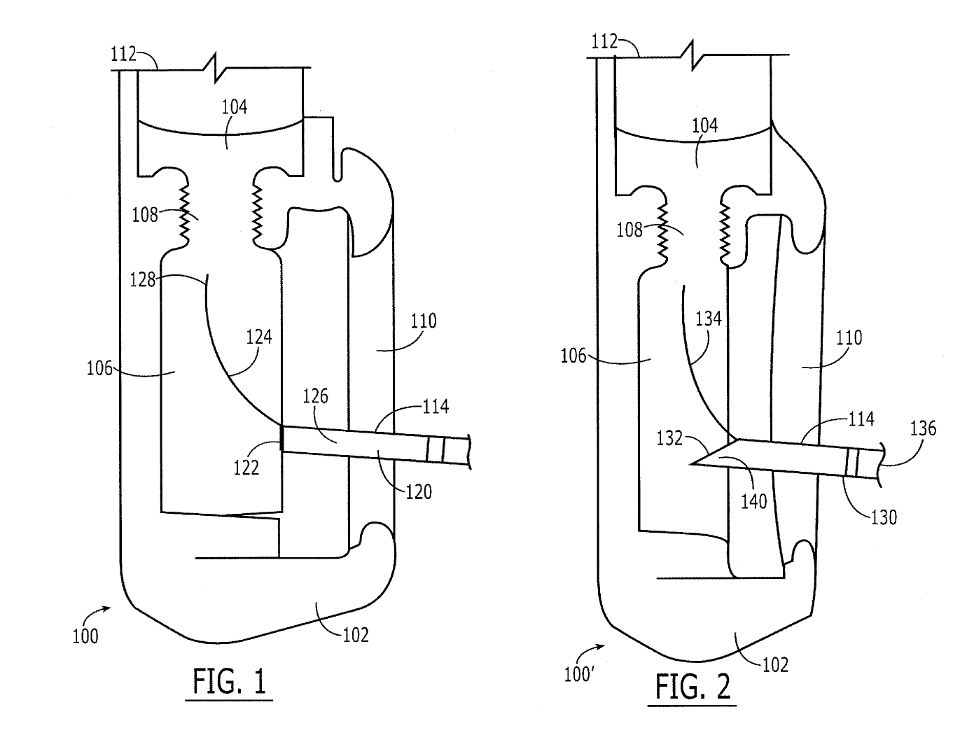

As shown in FIG. 1, an access system 100 according to an exemplary embodiment of the invention includes a port 102 with a connector 104 for fluid connection to a catheter 112. The port 102 includes a septum 110 closing a fluid chamber 106 and an outlet 104 connecting the fluid chamber 106 to the catheter 112. When the port 102 and catheter 112 are not used, the septum 110 seals the opening to the fluid chamber 106, to prevent fluid leakage from the catheter and the introduction of contaminants thereinto.

A needle or other syringe is inserted through the septum 110 to introduce fluids into or withdraw fluids from the catheter 112 via the fluid chamber 106. According to the invention, the needle is retained inside the port 102 against the back pressure exerted by the fluid in the fluid chamber 106 by an anchoring device. For example, the anchoring device may be deployed from a larger diameter hollow needle that pierces the septum 110.

According to the exemplary embodiment of FIG. 1, the access system 100 comprises an introducer needle 120 that pierces the septum 110 while minimizing damage to the septum material. As would be understood in the art, the size and tip shape of the introducer needle 120 and the material of the septum 110 are selected to ensure that the hole 114 formed in the septum 110 by the introducer needle 120 is re-sealed once the needle 120 has been removed by the natural bias of the material of the septum 110. For example, the introducer needle 120 may be a non-coring needle.

The anchoring device according to the present invention is inserted into the fluid chamber 106 via a lumen 126 extending through the introducer needle 120. As shown in FIG. 1, the anchoring device may comprise an anchoring needle 124 which, when deployed, extends distally beyond a squared off opening 122 of the introducer needle 120. The anchoring needle 124 preferably curves into the port 102 such that back pressure applied by the fluid therein presses the extended portion of the anchoring needle 124 against inner surface of the fluid chamber 106 and/or an inner surface of the septum 110. The fluid back pressure thus helps to retain the anchoring needle 124 in place by opposing a force tending to eject the anchoring needle 124 and the introducer needle 120 from the port 102 holding the introducer needle 120 in place within the port 102. A proximal end of the anchoring needle 124 is coupled to the introducer needle 120 so that the anchoring needle 124 may move distally and proximally relative to the introducer needle 120.

Once the anchoring needle 124 has been deployed, power injection procedures may be performed. In one exemplary embodiment, fluid is injected through the lumen 126 and out of the opening 122 into the fluid chamber 106 around an outer surface of the anchoring needle 124. Alternatively, the introducer needle 120 may be withdrawn and another injection needle may be advanced into the fluid chamber 106 over the anchoring needle 124, which acts as a guidewire. The power injection then takes place through a lumen of the injection needle 120 around the anchoring needle 124 or through the lumen of the injection needle 120 after the anchoring needle 124 has been pushed against a wall thereof.

As shown in FIG. 2, an introducer needle 130 according to a different embodiment of the invention includes a non-coring tip 140 cut at an angle relative to a longitudinal injection axis of the needle 130 to further reduce the possibility of damaging the septum 110 when it is pierced. In a preferred embodiment, the non-coring tip 140 is cut at an angle of 60° to 90° relative to the longitudinal axis of the needle 90°. In this embodiment, the anchoring needle 134 is advanced through the lumen 136 to exit into the fluid chamber 106 via an angled opening 132. The operation of the power injection apparatus is then substantially similar to that described above with reference to FIG. 1.

After the procedure has been completed, the anchoring needle 124 is withdrawn proximally until a distal tip thereof is received within the lumen 126 while the introducer needle 120 remains within the fluid chamber 106. The anchoring needle 124 may be completely withdrawn from the introducer needle 120 at this point or may remain within the lumen 126 as the introducer needle 120 is removed from the fluid chamber 106 allowing the septum 110 to seal the puncture 114. Any additional devices such as additional injection needles are also removed from the port 102 and the body at this point leaving the catheter 112 sealed within the body.

To prevent damage to a valve of a valved catheter or port, anchoring needle 124 of the present invention may be extended to maintain such a valve in an open position during the procedure. After the procedure has been completed, the anchoring needle 124 is withdrawn allowing the valve to close under its natural bias. These valves are often located near the port 102 (e.g., in the outlet 108). Thus, for a given application, a length of the anchoring needle 124 is selected to enable a distal end 128 thereof to be advanced distally beyond the distal tip of the introducer needle 120 across the fluid chamber 106 and through the valve. The distal end 128 is preferably formed as a smooth blunt shape to minimize the chance of damage to the valve. In the case of a PASV, the distal end 128 is passed through a slit in the membrane pushing opposite edges of the slit away from one another to hold the valve open. Fluid from the power injection then passes through the PASV without unduly stressing the membrane. As would be understood by those skilled in the art, the anchoring needle 124 is preferably shaped so that, as the distal end 128 leaves the confinement of the lumen 126, a bias of the material of the anchoring needle 124 bends the distal end 128 to extend laterally away from an axis of the introducer needle 120. The physician may then rotate the anchoring needle to point the distal end 128 toward the valve (e.g., toward the outlet of the port 102). Those skilled in the art will understand that the proximal end of the anchoring needle 124 may include visual or tactile indications of the orientation of the distal end 128 facilitating aiming of the distal end 128 toward the valve. Alternatively, the anchoring needle 124 and the lumen 126 may be shaped so that only one insertion orientation is possible. For example, as would be understood by those skilled in the art, a key or other shape protruding from the anchoring needle 124 may mate with a corresponding recess in the lumen 126 so that an orientation of the anchoring needle 124 relative to the introducer needle 120 will always be known.

In addition, the physician may control a height at which the distal end 128 traverses the fluid chamber 106 by adjusting a height of the distal end of the introducer needle 120 relative to a base of the fluid chamber 106 to aim the distal end 128 toward a desired portion of the valve. For example, for a PASV with a substantially central slit, the physician may wish to engage a center of the membrane or an area a predetermined distance from the center. To do this, the physician may insert the introducer needle until is contacts the base of the fluid chamber 106 and then withdraw the introducer needle 120 by a desired distance so that the distal end 128 exits the introducer needle 120 at the desired height. Then, as the anchoring needle 124 is advanced distally through the lumen 126, the distal end traverses the fluid chamber toward the valve along a desired trajectory which results in the distal tip 128 engaging the valve at the desired height.

The present invention has been described with reference to specific exemplary embodiments. Those skilled in the art will understand that changes may be made in details, particularly in matters of shape, size, material and arrangement of parts. Accordingly, various modifications and changes may be made to the embodiments. The specifications and drawings are, therefore, to be regarded in an illustrative rather than a restrictive sense.

What is claimed is:

1. A power injection system, comprising:
an introducer needle including a lumen extending therethrough to a non-coring distal tip; and
an anchoring device extendable laterally from the introducer needle distal tip, the anchoring device including a port contacting surface engaging one of an inner surface of the port and an inner surface of a septum to anchor the introducer needle within a subcutaneous port.

2. The power injection system according to claim 1, wherein the anchoring device comprises a flexible needle having a distal portion coupled to a proximal portion, the anchoring needle being formed so that, when the distal portion is not constrained within the introducer needle lumen, the distal portion bends away from the introducer needle along a predetermined trajectory.

3. The power injection system according to claim 2, wherein a distal end of the anchoring needle is formed as a smooth blunt shape for engaging and opening a valve located within one of the catheter port and a catheter coupled to the catheter port.

4. The power injection system according to claim 1, wherein the introducer needle lumen extends to an opening in a distal tip of the introducer needle and wherein the opening extends in a plane substantially perpendicular to a longitudinal axis of the introducer needle.

5. The power injection system according to claim 1, wherein the introducer needle lumen extends to an opening in a distal tip of the introducer needle and wherein the opening extends in a plane angled with respect to a longitudinal axis of the introducer needle.

6. The power injection system according to claim 5, wherein the angle is between 60° and 90°.

7. The power injection system according to claim 17 wherein the introducer needle lumen and the anchoring device are sized so that, when the anchoring device extends through the introducer needle lumen, power injection fluid may be injected through an annular space surrounding the anchoring device.

8. The power injection system according to claim 17 wherein a proximal portion of the anchoring device includes an orientation indicator enabling a user to determine a direction at which the distal portion of the anchoring device projects laterally from the introducer needle.

9. The power injection system according to claim 8, wherein the orientation indicator includes one of a visible marking and an asymmetric shape.

10. The power injection system according to claim 1, wherein the anchoring device is shaped to mate with the introducer needle lumen in only one desired orientation.

11. The power injection system according to claim 10, wherein the anchoring device includes one of a protrusion and a key and wherein the introducer lumen includes one of a key and a protrusion mating with the one of a protrusion and a key of the anchoring member.

12. The power injection system according to claim 17 further comprising a locking device for locking a position of the anchoring device relative to the introducer needle after the anchoring device has been deployed to a desired position within a port.

13. A method for performing power injections, comprising:
inserting an introducer needle into a fluid chamber of a catheter port via a septum of the port, the introducer needle defining an introducer needle lumen extending therethrough to a distal tip;
extending an anchoring device from a distal end of the introducer needle lumen;
advancing the anchoring device laterally to engage an inner surface of one of the port and the septum; and
power injecting fluid into the catheter port.

14. The method according to claim 13, wherein the fluid is power injected via the introducer needle lumen.

15. The method according to claim 13, wherein the distal tip comprises an angled non-coring tip.

16. The method according to claim 15, wherein the angle is between 60° and 90°.

17. The method according to claim 13, wherein the anchoring device is inserted through the introducer needle lumen.

18. The method according to claim 13, further comprising advancing a distal end of the anchoring device through the port to engage and open a flow control valve in one of the port and a catheter attached thereto.

19. The method according to claim 18, further comprising orienting the anchoring device toward the valve using an indicator on a proximal end of the anchoring device.

20. The method according to claim 19, wherein the indicator is one of a visible marker and a tactile indicator.

21. The method according to claim 13, further comprising mating an orienting structure of the anchoring device with a corresponding orienting structure of the introducer needle to ensure that the anchoring device remains in a desired angular orientation relative to the introducer needle.

22. The method according to claim 13, further comprising locking the anchoring device to the introducer needle after the anchoring device has reached a desired position withing the port.

23. The method according to claim 13, further comprising withdrawing the anchoring element into the introducer needle lumen and removing the introducer needle from the catheter port after the power injection has been completed.
